(19) United States
(12) Patent Application Publication
(10) Pub. No.: US 2007/0003483 A1
(43) Pub. Date: Jan. 4, 2007

(54) Method of evaluating organ function

(75) Inventor: Makoto Suematsu, Tokyo (JP)

Correspondence Address: DARBY & DARBY P.C., P. O. BOX 5257, NEW YORK, NY, 10150-5257, US

(73) Assignee: Oxygenix Co., Ltd., Tokyo (JP)

(21) Appl. No.: 10/573,062

(22) PCT Filed: Sep. 28, 2004
(86) PCT No.: PCT/JP04/14566

§ 371 Date: Mar. 21, 2006

Related U.S. Application Data
(60) Provisional application No. 60/506,506, filed on Sep. 29, 2003.

(51) Int. Cl. A61K 49/00; C12Q 1/00

(52) U.S. Cl. 424/9.6; 435/4

(57) ABSTRACT

The present invention provides a method of analyzing organ or tissue injury, comprising the following steps of: (a) labeling the organ or tissue with dye; (b) obtaining multiple indices involving xenobiotic metabolism and/or cell condition of said organ or tissue; and (c) analyzing the organ or tissue injury from said indices.

### TECHNICAL FIELD

The present invention relates to a method of evaluating organ function. More specifically, the present invention relates to a method for analyzing visually and/or for determining quantitatively multiple indices with microanatomical orientation vascular system and/or excretion pathways.

### BACKGROUND ART

Although use of University of Wisconsin solution has improved mean preservation time for liver transplantation, the incidence of primary graft nonfunction and initial poor function still persist (1-3). The clinical incidence of such a dysfunction and the resultant loss of graft survival depend on the storage time (1-4). Reperfusion injury is the key event leading to the graft failure after prolonged cold ischemia (5-9). During the storage, hepatocytes swell and form blebbing (6-8). Upon reperfusion, however, these changes in the parenchymal cells were restored without leading to their irreversible injury (6-8). On the other hand, sinusoidal endothelial cells lose their viability and Kupffer cells are activated (6-10). According to previous studies using rat liver grafts stored in UW solution, the critical storage time when the changes in sinusoidal cells occur is longer than 16 hrs (6, 7). In these grafts, the cells were damaged to cause platelet trapping (10), fibrin deposition (11), and leukocyte margination (12). In grafts stored for shorter duration, hepatic ATP content was reported to be well recovered after reperfusion, suggesting that parenchymal cells are viable (13, 14).

Apart from these data, it has not been carefully examined if hepatocytes lose their functions without displaying irreversible injury and then interfere with the graft function as a whole. Although biliary output, clearance of taurocholate and bromosulfophtalein were measured in previous studies (13-15), they failed to demonstrate such functional alterations in hepatocytes even when the storage time was extended to 18 hrs (13). Besides these studies, little information has been available as to alterations in the ability of the post-cold ischemic grafts to excrete bile constituents. Such indices include the ability of hepatocytes to yield the osmotic driving force for bile formation, and to excrete bile salts or organic anions; thus, excretion of glutathione and bilirubin could serve as a marker detecting early hepatocellular changes in the grafts. The ability to excrete organic anions could determine the efficiency of the graft to detoxify xenobiotics and the severity of post-cold ischemic hyperbilirubinemia, a risk factor for allograft dysfunction in clinical transplantation (2, 16).

### DISCLOSURE OF INVENTION

It is an object of the present invention to provide a method of evaluating organ function. Based on the above findings, the present inventor continued further investigations.

In attempt to determine such an ability of post-cold ischemic grafts, we have herein examined changes in constituents of bile samples as a function of the storage time, and revealed impaired excretion of glutathione and bilirubin as an early event on hepatocytes. This event turned out to be the result of cytoplasmic re-localization of multidrug resistance associated protein 2 (Mrp2), an ATP-dependent transporter for biliary excretion of the organic anions. Our results suggest that the function of this transporter is impaired whereas the grafts apparently maintain their overall energy charges without showing any notable hepatocellular damages. Furthermore, mechanisms for such a change in hepatocytes appear to involve thromboxane synthesis in Kupffer cells in grafts exposed to a rather short duration of cold ischemia; i.e. 8 hrs.

The present application provides the following inventions:

(1) A method of analyzing organ or tissue injury, comprising the following steps of:
(a) labeling an organ or a tissue with dye;
(b) obtaining multiple indices involving xenobiotic metabolism and/or cell condition of said organ or tissue; and
(c) analyzing the organ or tissue injury from said indices.

(2) The method of (1), wherein the organ or tissue is at least one selected from the group consisting of liver, kidney, lung, pancreas and gastrointestinal tracts.

(3) The method of (1) or (2), where the step (b) further comprises a step of obtaining microanatomical orientation of vascular system and/or excretion pathways.

(4) The method of any one of (1) to (3), wherein the analysis is carried out visually and/or quantitatively.

(5) The method of any one of (1) to (4), wherein the cell condition is at least one selected from the group consisting of cell viability, cell injury, molecular transport, and mitochondrial function.

(6) A method of evaluating drug toxicity, comprising the following steps of:
(a) labeling an organ or a tissue with dye;
(b) applying a test drug to said organ or tissue;
(c) obtaining multiple indices involving xenobiotic metabolism and/or cell condition of said organ or tissue;
(d) analyzing the organ or tissue injury from said indices; and
(e) evaluating whether or not the drug have a toxicity to said organ or tissue.

(7) The method of (6), wherein the organ or tissue is at least one selected from the group consisting of liver, kidney, lung, pancreas and gastrointestinal tracts.

(8) The method of (6) or (7), where the step (b) further comprises a step of obtaining microanatomical orientation of vascular system and/or excretion pathways.

(9) The method of any one of (6) to (8), wherein the analysis is carried out visually and/or quantitatively.

(10) The method of any one of claims 6 to 9, wherein the cell condition is at least one selected from the group consisting of cell viability, cell injury, transport of molecules in and around cells, and generation of biologically active compounds, blood flow, and tissue oxygenation.

### BRIEF DESCRIPTION OF DRAWINGS

FIG. 1 shows a time courses of the bile output of liver grafts that have undergone cold preservation followed by reperfusion. Open circles denote the data from non-ischemic control livers. Open, shaded, and closed squares indicate the data from grafts exposed to 8, 16, 24 hr-cold storage, respectively. Closed triangles indicate the data from the 48-hr storage grafts. Values are mean±SE of 5 separate experiments. TC (+) and TC (−): data collected in the presence and absence of sodium taurocholate at 30 μmol/L. R: the onset of reperfusion. Note that both bile salt-independent and -dependent outputs were decreased in the 16-hr preserved grafts. *P<0.05 as compared with the data from control. †P<0.05 as compared with the data from 16-hr grafts. #P<0.05 as compared with the data from 24-hr grafts.

FIG. 2 shows an Effects of duration of cold ischemia on biliary concentrations of bile constituents. Data of bile constituents were collected at 20 min after the onset of reperfusion. A: bile salts (Panel A). B: phospholipids. C: concentrations of reduced glutathione (GSH) in bile. D: hepatic content of GSH in the control and 16-hr stored grafts measured before and 20 min after reperfusion. E: concentrations of bilirubin (BR)-IXα in bile. F: hepatic content of BR-IXα in the control and 16-hr stored grafts measured before and 20 min after reperfusion. Values are mean±SE of 5-7 separate experiments. *P<0.05 as compared with the data from control livers. #P<0.05 as compared with the data from the 16-hr group. †P<0.05 as compared with the data from the control grafts exposed to 20-min reperfusion.

FIG. 3 shows alterations in dynamics of hepatocellular carboxyfluorescein (CF) excretion into bile canaliculi in cold ischemia-reperfused grafts. A: Representative pictures of the canalicular CF excretion captured before (Basal loading), and 10 min and 25 min after removal of probenecid. Note disruption of honeycomb patterns of bile canalicular networks in the 24-hr cold ischemic-reperfused graft (arrow). Color bar indicates the fluorescence intensities calibrated with known concentrations of CF. Bar: 30 μm. B: Differences in reperfusion-induced disruption of bile canalicular networks as judged by the density of CF-filled polygons in the grafts stored for varied duration of cold ischemia. *P<0.05 as compared with the data from control livers. C: Initial hepatocellular CF concentrations showing comparable CF loading among groups. Values are mean±SE of 5 separate experiments.

FIG. 4 shows in vivo quantitative analyses of Mrp2 function by visualizing bile canalicular excretion of carboxyfluorescein (CF). A: Representative series of pictures showing the dye excretion from individual hepatocytes. Upper; images from a graft perfused in the presence of 1.5 mM probenecid (PB(+)). Lower, images captured after the removal of probenecid (PB(−)). Note time-dependent reduction of the fluorescence in the cells followed by condensation and disappearance of the dye in surrounding bile canaliculi. The dye retention in the cells should be noted. B: The decay of hepatocellular CF fluorescence. Hepatocellular CF concentrations in the grafts treated with (closed circles) or without (open circles) probenecid, were plotted semi-logarithmically against the time so that a straight line represents an exponential curve. Inset: the calibration curve indicating the relationship between concentrations of CF and 8-bit gray levels. [CFapp]: apparent concentrations of CF. CF concentrations were linearly related to gray levels at the concentrations smaller than 3 μmol/L (r²=0.996, p<0.05). C: Differences in half-life time (T_{1/2}) of CF exclusion from hepatocytes. Values are mean±SE of 5 separate experiments in each group. *P<0.05 as compared with the data from control livers. EHBR: grafts isolated from Eisai hyperbilirubinemia rats. PB: grafts perfused with 1.5 mM probenecid.

FIG. 5 shows alterations in the ability of liver grafts to excrete carboxyfluorescein (CF) into bile upon cold ischemia-reperfusion. A: Differences in time course of the biliary CF excretion in grafts exposed to varied lengths of cold ischemia. Open circles: control grafts perfused right after removing 1.5 mM probenecid, an Mrp2 inhibitor. Open and shaded squares: grafts undergoing 8- and 24-hr cold storage followed by reperfusion in the absence of probenecid, respectively. Shaded circles: grafts normoperfused in the presence of probenecid. Closed circles: normoperfused grafts isolated from Eisai hyperbilirubinemia rats. Values are mean±SE of 5 separate experiments. T₀: time when probenecid was removed from the perfusate. Inset: a linear relationship between the CF concentration and fluorescence intensities. B: Alterations in relative CF concentrations in bile collected at varied duration of reperfusion, and effects of the depletion of Kupffer cells (KC). Left: Differences in the decay of biliary CF excretion between the control (0 hr, broken lines) and 8-hr-stored liver grafts (8 hrs, solid lines). Right: Effects of KC depletion by intravenous liposome-encapsulated dichloromethylene diphosphonate (LDD). Open circles: control (0 hr, broken lines). Open squares: 8-hr-stored grafts (8 hrs, solid lines). Data represent means±SE of measurement from 4 separate experiments. *P<0.05 as compared with the decays of CF exclusion in control livers. C: Effects of KC depletion [KC(−)] by LDD and/or treatment with OKY-046 (OKY), an inhibitor of thromboxane A₂ synthase, on lengthening T_{1/2} values in the 8-hr cold storage livers. IM: indomethacin. Concentrations of OKY and IM in the storage and rinse solutions were 240 and 28 μmol/L, respectively. Note that an inhibitory action of OKY disappears in the KC-depleting grafts. Values are mean±SE of 5-6 separate experiments. *P<0.05 as compared with the data from control livers. †P<0.05 as compared with the data in the 8-hr stored grafts.

FIG. 6 shows disruption of intracellular distribution of Mrp2 in liver grafts exposed to 8-hr cold ischemia and 60-min reperfusion (8 hr/R), and effects of Kupffer cell (KC)-depleting procedure [KC(−)] or treatment with OKY-046 (OKY). A: Western blot analyses and immunoprecipitation of Mrp2 by the anti-acrolein monoclonal antibody 5F6. M: molecular marker. B: Immunofluorescence analyses of Mrp2 distribution. Left; single staining with the anti-Mrp2 monoclonal antibody (M₂III-6) labelled with phycoerythrin. Bar: 30 μm. Right; double immunostaining with the FITC-labelled ZO-1 antibody and the phycoerythrin-labelled M₂III-6 antibody. Bar: 10 μm. C. Semiquantitative analyses of hepatocellular Mrp2 localization. % I-Mrp2(cyt/bc); cytoplasmic intensities of Mrp2-associated immunoreactivities versus those measured at bile canaliculi. Values are mean±SE of measurements in 40-60 hepatocytes/graft from 4 separate livers. *P<0.05 as compared with the data from control livers. †P<0.05 as compared with the data collected from the 8 hr/R-KC (+) group.

### BEST MODE FOR CARRYING OUT THE INVENTION

Hereinbelow, the present invention will be described in more detail.

Although Kupffer cells (KC) have been thought to play a crucial role in post-cold ischemic hepatocellular injury, their roles in non-necrotic graft dysfunction remain unknown. This study aimed to unravel such roles of KC in the post-cold ischemic liver grafts. Rat livers treated with or without liposome-encapsulated dichloromethylene diphosphonate, a KC-depleting reagent, were stored in University of Wisconsin solution at 4° C. for 8-24 hrs and reperfused under monitoring biliary output and constituents. An ability of hepatocytes to excrete bile was assessed through laser-confocal microfluorography in situ. Cold ischemia-reperfused grafts decreased their bile output significantly at 8 hrs without any notable cell injury. This event coincided with impaired excretion of glutathione and bilirubin-IXα, suggesting retarded transport of these organic anions. Mechanisms involved intracellular re-localization of multidrug resistance protein-2 (Mrp2). Kinetic analyses for biliary excretion of carboxyfluorescein, a fluoroprobe excreted through this transporter, revealed significant delay of the dye excretion from hepatocytes into bile canaliculi. The KC-depleting treatment significantly attenuated this decline in biliary anion transport mediated via Mrp2 in the 8-hr cold ischemic grafts through re-distribution of Mrp2 from the cytoplasm to the canalicular membrane. Furthermore, mechanisms appeared to involve thromboxane A2 synthase in KC, since improving effects of blocking this enzyme on CF excretion and on cytoplasmic internalization of Mrp2 disappeared in the KC-depleting grafts. In conclusion, these results suggest that KC activation is an important determinant of non-necrotic hepatocellular dysfunction, jeopardizing homeostasis of the detoxification capacity and organic anion metabolism of the post-cold ischemic grafts.

In the present specification, some abbreviations are used: BC, bile canaliculi; BR-IXα, bilirubin-IXα; CF, 5-carboxyfluorescein; CFDA, 5-carboxyfluorescein diacetate; EHBR, Eisai hyperbilirubinemia rats; GSH, reduced glutathione; KC, Kupffer cells; LDD, liposome-encapsulated dichloromethylene diphosphonate; Mrp2, multidrug resistance protein 2; TXA₂, thromboxane A₂.

In one embodiment of the present invention, a method of analyzing organ or tissue dysfunction and/or injury is provided. The method comprises the following steps:

(a) labeling the organ or tissue with one or two dyes;

(b) obtaining multiple indices indicating cell and/or organ functions involving molecular transport and excretion, xenobiotic metabolism and/or cell condition of said organ or tissue; and

(c) analyzing the organ or tissue injury from said indices.

In the present invention, organ or tissue injury means necrosis, apoptosis or disruption of cytoplasmic membrane.

In order to label the organ or tissue under living conditions, conventional methods using a dye (e.g. fluorescent dye, rhodamines or fluorescein salts or propidium iodide) may be employed. For example, these dyes may be loaded with intravenous injection or intraperitoneal injuection in experimental animals, or with intravascular perfusion with their controlled concentrations into the isolated perfused organ preparations in liver, kidney, pancreas, gastrointestinal tracts or lung.

Multiple indices means cell viability, mitochondrial membrane potentials, oxygen free radicals, transport of anions or cations across the cell membrane, cytoplasmic membrane potential, and cell death including apoptosis or necrosis. Examples of the mutiple indices include, but are not limited to xenobiotic metabolism and cell viability.

Xenobiotic metabolism means metabolism and/or catabolism of any types of exogenous reagents or medicines that are degraded in the body.

Cell condition means any types of functional states of living cells including molecular transport (e.g. molecular transport in and aroudnd cells), membrane potential, mitochondrial function, generation of biologically active compounds (generation of bioactive species), blood flow and tissue oxygenation, and uptake and excretion of anions and cations, and those of cells losing their viability such as necrosis or apoptosis. Examples of cell condition include, but not limited to cell viability and cell injury.

In one embodiment of the present invention, a term “microanatomical orientation” means geographic relationship between cells and their surrounding structures such as microvascular systems and the interstitial space involving lymphatic systems and/or connective tissues (e.g. bile canalicular system in the liver). Thus, examples of the microanatomical orientation include, but not limited to bile canalicular networks in liver.

A term “vascular system” means macro and microcirculatory systems involving conducting vessels, arterioles, capillaries and venules.

A term “excretion pathway” means pathways that allow intracellular molecules to be excreted into the extracellular space.

In the present invention, a monitoring of whole-organ functional parameters may be employed to investigate organ or tissue injury visually or quantitatively. A term “visually” means capture of intravital images that include quantitative information of biological activities. A term “quantitatively” means determinating values that indicate biological activities. The whole-organ functional parameters include, but not limited to bile output, determination of bile constituents such as bile acids, phospholipids, cholesterol and bicarbonate.

In another embodiment of the present invention, a method of evaluating drug toxicity is provided. In the present invention, the following steps are included:
(a) labeling an organ or a tissue with dye;
(b) applying a test drug to said organ or tissue;
(c) obtaining multiple indices involving xenobiotic metabolism and/or cell conditions of said organ or tissue;
(d) analyzing the organ or tissue injury from said indices; and
(e) evaluating whether the drug have a toxicity to said organ or tissue.

In the present invention, a drug toxicity means any types of harmful actions of reagents or medicines on living cells involving inhibition of enzymes or transporter functions. Examples of the drug include, but not limited to inhibitor of ABC transporter (e.g. Probenecid as an inhibitor of mrp2, cyclosporine, adriamycin, cysplatinum, cimetidine, etc.).

A word “applying a test drug” means contacting a drug of interest to organ or tissue. The drug toxicity may be evaluated by analyzing the organ or tissue injury from some indices. For example, when indices for function of any ABC transporter are changed, then it is evaluated that the drug (e.g. ABC transporter) has a toxicity to the organ or tissue. When the indices are kept stable, then it is evaluated that the drug (e.g. ABC transporter) has not a toxicity to the organ or tissue.

The present invention provides evidence that the impaired ability of hepatocytes to execute the Mrp2-dependent excretion of organic anions accounts for an early event indicating the graft dysfunction caused by organ or cell injury such as cold-ischemia followed by the short duration of reperfusion. This change on hepatocytes was subtle and non-necrotic, but critical enough to cause the imbalance between the cellular generation and excretion of glutathione and bilirubin at the level of whole grafts. Extending the duration for cold ischemia up to 16 hrs induced dysfunction of bile canaliculi as characterized by disappearance and dilation of them, while their polygonal networks were kept intact unless the duration of cold storage exceeded 8 hrs. To our knowledge, it remains unknown whether such a non-necrotic dysfunction of hepatocytes exposed to a relatively short period of cold ischemia could be mediated by post-ischemic responses of sinusoidal cells involving KC. As hereby shown, the reduced ability to excrete organic anions via Mrp2 was completely restored by depleting KC, suggesting involvement of this sinusoidal cell in mechanisms for the dysfunction.

The impairment of Mrp2-mediated transport in the 8-hr post cold-ischemic grafts results from cytoplasmic re-localization of this transporter from canalicular membrane but neither from disruption of bile canalicular networks nor from oxidative modification of the transporter by itself; this is consistent with our previous observation that the 8-hr cold ischemia and reperfusion does not show any notable oxidative stress in the liver grafts (14). Alterations in cAMP, a determinant for bile canalicular sorting of Mrp2 (32, 40, 41), are unlikely to play a role in the KC-mediated dysfunction, because its content did not differ irrespective of the presence of KC. Hepatocellular content of ATP are another determinant for the transporter function, but likely to play little role, if any, in the mechanisms, since any differences were notable between the KC-depleting and control grafts having undergone 8-hr cold ischemia. Since the KC depletion did not alter the ability of Mrp2 to excrete organic anions in normal livers, such an alteration of the transporter function in the grafts appears to result from responses of KC that cannot be triggered unless the graft had undergone cold ischemia-reperfusion. Although detailed mechanisms remained unknown, the present results suggest involvement of TXA₂ synthase, the enzyme responsible for TXs, a major class of prostanoids released from KC (37, 38). Observation that the preventive effect of the enzyme inhibitor was completely cancelled in the KC-depleted grafts led us to suggest that KC constitutes a major source of TXs that trigger internalization of Mrp2 into the cytoplasm of hepatocytes. Although TXA₂ has been thought to exert potent biological actions on various types of cells, previous studies provided evidence that TXB₂, a relatively stable metabolite of TXA₂, is able to activate non-lysosomal proteinases and thereby triggers bleb formation of primary cultured hepatocytes (42). Thus, further mechanisms by which KC-derived TXs cause hepatocellular function should be necessary.

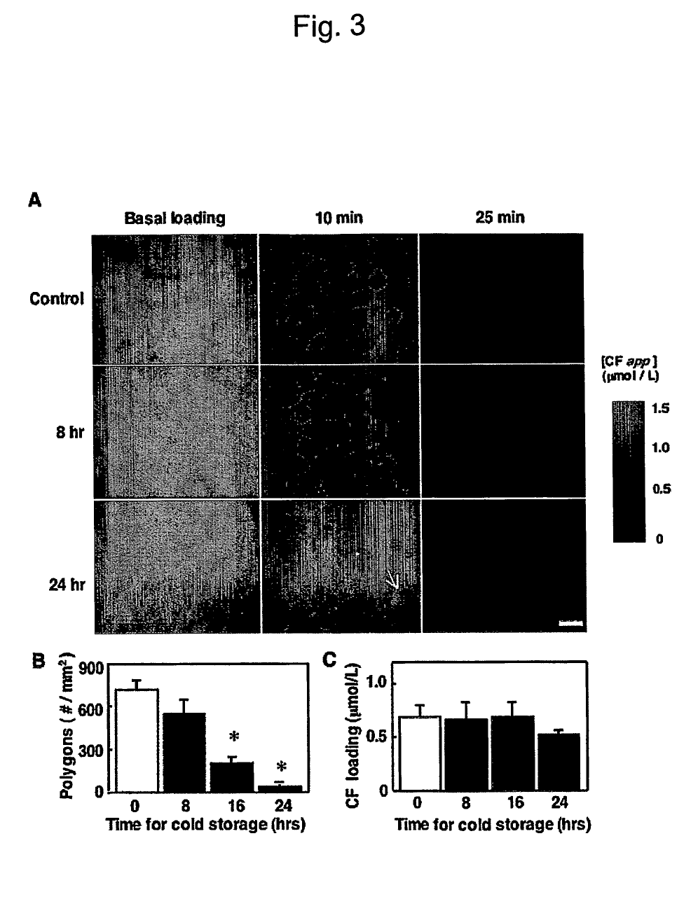

The newly developed method of the dye exclusion analyses from grafts preloaded with controlled amounts of CF revealed that re-localization of Mrp2 occurs at hepatocellular levels and results in significant deterioration of the whole-graft function. As seen in FIG. 3, the 8-hr storage significantly reduced biliary glutathione excretion without showing any change in the tissue content, if any. Since this organic anion serves as the major substance yielding the osmotic driving force for bile acid-independent bile formation, its reduction in bile could result in a decrease in the output. This notion is also consistent with our observation that 8-hr stored grafts displayed a significant reduction of the output.

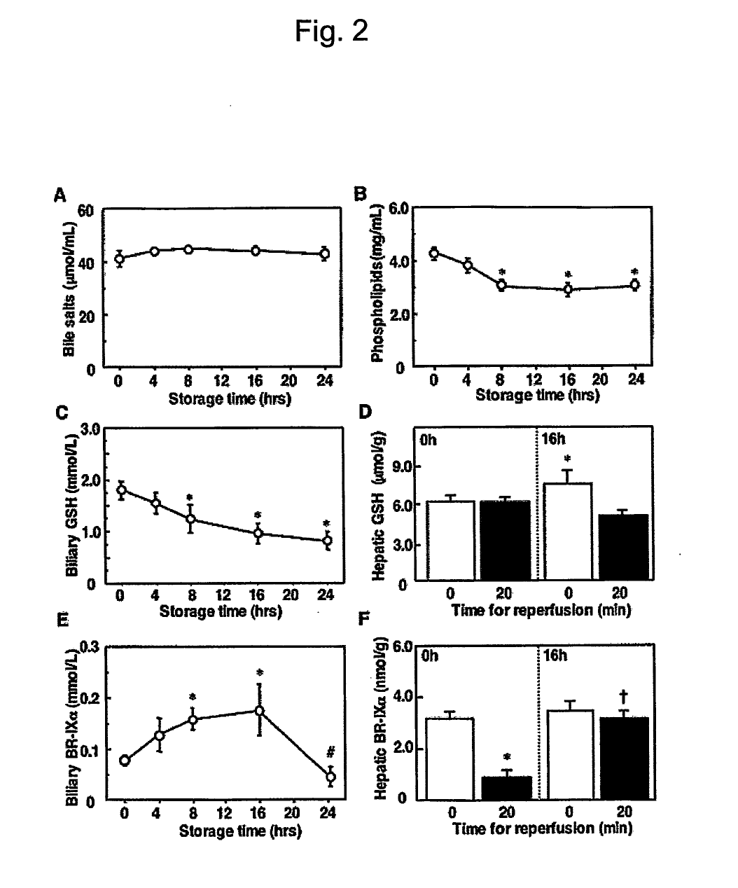

In this context, the imbalance between endogenous generation and biliary excretion of BR-IXα in the grafts is of great interest in the current study. As seen in FIG. 2, the control liver can excrete approximately 75% of endogenous BR-IXα into bile within 20 min of perfusion, consistent with our previous studies (24). On the other hand, such a rapid elimination of the bile pigment did not occur in the 16-hr cold ischemic grafts. As judged by biliary concentrations of BR-IXα (FIG. 2E), the absolute amounts of the pigment were elevated but never decreased as compared with the non-cold ischemic control grafts. Since amounts of BR-IXα released into circulation were negligible (data not shown), these results suggest that the cold ischemic grafts synthesize greater amounts of the pigment during the initial 20-min reperfusion than those expected from their capacity to excrete it into bile. This notion is in good agreement with our observation that the graft induces heme oxygenase-1, the stress-inducible enzyme for heme degradation (43). This event is of pathophysiological importance from a viewpoint of anti-oxidative stress responses of the post-cold ischemic grafts. We have recently reported that low-dose bilirubin can ameliorate oxidative stress and thereby protect the post-cold ischemic liver grafts, although its excess dose is obviously harmful (31, 43). In the grafts exposed to cold ischemia, reperfusion could cause two important events that critically dictate hepatic bilirubin metabolism; increased heme degradation and retardation of BR-IXα through Mrp2. Thus, combined actions of these two events could result in accumulation of this antioxidant sufficient enough to protect hepatocytes, while their prolonged effects lead to hepatocellular damages and hyperbilirubinemia in the later period of reperfusion.

KC serves as a potent generator of eicosanoids, while hepatocytes and ATP binding cassette transporters expressed on their membrane help their degradation and excretion, respectively (38, 39). On the other hand, anti-oxidant organic anions such as glutathione and bilirubin share Mrp for their excretion into bile in the post-cold ischemic grafts. Thus, the balance between KC-mediated synthesis of eicosanoids and their removal from hepatocytes could determine redistribution of the anti-oxidant anions in and around hepatocytes and thereby dictate functional outcome of liver transplantation. KC-mediated remodeling of Mrp-mediated organic anion transport deserves further studies provided that quantitative information on intra- and inter-cellular kinetics of glutathione, and BR-IXα becomes available to examine if KC-yielded thromboxanes could serve as an early alert mechanism against subsequent oxidative stress on the graft.

### EXAMPLE

Hereinbelow, the present invention will be described in more detail with reference to the following Examples. It should be noted that the technical scope of the present invention is not limited by these Examples.

Materials and Methods

Animal Preparation

The experimental protocols herein described were approved by our institutional guidelines provided by the Animal Care Committee of Keio University School of Medicine. Male Wistar rats (220-260 g, CLEA Japan, Tokyo) and Eisai hyperbilirubinemia rats (EHBR) (220-260 g, Sankyo Inc., Tokyo) that had been allowed free access to laboratory chow and tap water were fasted 24 hrs before experiments. Livers of these rats were perfused ex vivo with the oxygenated Krebs-Henseleit buffer as the base-line perfusate (17, 18), and stored in the University of Wisconsin solution at 4° C. for desired lengths of time (14). When necessary, rats were pretreated with an intravenous injection of liposome-encapsulated dichloromethylene diphosphonate (LDD) at 24 hrs prior to preparation of the ex-vivo liver perfusion for the cold storage according to our previous studies (14, 19). As described previously, this procedure eliminated Kupffer cells almost completely, as judged by immunohistochemistry (20). After the cold storage, the grafts were gently rinsed with a transportal injection of 40 mL of the lactated Ringer solution and perfused with the oxygenated buffer in the presence or absence of sodium taurocholate at 30 μmol/L at a constant flow (32 mL/min) in a single-pass mode (14, 21). For some experiments, either OKY-046, an inhibitor of thromboxane A₂ (TXA₂) synthase, or indomethacin (IM), an inhibitor of cyclooxygenase, was added in UW solution as well as in the rinse solution at desired concentrations (22).

Determination of Bile and Tissue Constituents

Bile samples were used to determine concentrations of total bile salts, phospholipids, reduced glutathione (GSH) and bilirubin-IXα (BR-IXα) (23, 24). BR-IXα was determined by an enzyme-linked immunosorbent assay using 24G7 (24). This monoclonal antibody can recognize BR-IXα, the terminal heme-degrading product generated specifically through the HO reaction as described earlier (24, 25). Activities of lactate dehydrogenase (LDH) were measured as described earlier (17). Adenosine triphosphate (ATP) in the liver grafts was determined by the luciferrin/luciferase method as described elsewhere (14, 21). Cyclic AMP in the grafts was determined by an enzyme-linked immunosorbent assay (Biotrak™ system, Amersham Biosciences, Buckinghamshire).

Analyses of Biliary Excretion Rates of Carboxyfluorescein

Carboxyfluorescein (CF) is an organic anion which excreted from various cells through Mrp2 (26, 27). The ester precursor of this dye, CF diacetate (CFDA) was loaded transportally into the livers at 50 nmol/L for 10 min in the presence of 1.5 mmol/L probenecid, a potent inhibitor of Mrp2 (26, 28). This reagent can enter hepatocytes and is hydrolyzed by esterase into CF to be excreted into bile (14, 17, 29). After the 10-min CFDA loading, the liver was perfused with the probenecid-free buffer to trigger the excretion of CF into bile. In the cold ischemic groups, the stored grafts were loaded with the CFDA containing buffer for 10 min in the presence of probenecid, and followed by removal of probenecid and subsequent reperfusion for 50 min. Bile samples collected from these preparations were deep-frozen until the fluorescence measurements were carried out using a 96-well multi-channel fluorescence spectrophotometer. The measurements were performed under epi-illumination at 440 nm, the isosbestic wavelength of the dye which yields fluorescence at 510 nm without interference with pH values of the samples (26). The concentration of CF in samples was calibrated with known concentrations of CF dissolved in phosphate buffer saline. As seen later in Results, CF concentrations appeared to decline exponentially with time. With this assumption, biliary CF lifetimes were determined as the T_{1/2} of the exponential decay. Thus, this method is insensitive to the initial amounts of CF loaded into the perfused liver.

In situ Visualization of Hepatocellular CF Exclusion.

Liver grafts loaded with CF using the aforementioned protocols were observed through intravital laser confocal microfluorography as described previously (14, 20, 30). As shown later in Results, CF was notably loaded into hepatocytes in the presence of probenecid. Upon removal of the reagent, the dye was immediately excluded from hepatocytes, excreted into bile canaliculi (BC) to display honeycomb networks, and finally disappeared from the parenchyma. To examine if the dye exclusion depends on function of Mrp2, some grafts were reperfused with the buffer containing 1.5 mmol/L probenecid. The laser confocal microfluorographs were captured by an inverted-type microscope (Diaphot 300, Nikon/Sankei) equipped with intensified CCD camera (C5810, Hamamatsu Photonics) and multi-pinhole laser confocal processor (CSU-10, Yokogawa Electric Co.). All microfluorographs were digitally processed into 8-bit gray level images. To calibrate the fluorescence intensities, known concentrations of CF were prepared in vitro and the images were captured under the identical optical parameters of the camera. Gray levels in hepatocytes were measured by variable square window (2×2 μm²) using digital image processor (18, 31). At least 10 different hepatocytes in the microscopic fields of interests were analyzed in a single experiment. Assuming that fluorescence intensities measured at the liver surface is identical to those measured in the solution, gray levels were converted to apparent CF concentrations using the calibration line, being designated as CFapp.

We also conducted morphometry to examine structural changes in BC networks as an index of hepatocellular damages. As shown later in Results, normally functioning hepatocytes were characterized by polygonal CF filling in surrounding BC, while those damaged were judged by partial disappearance of the surrounding BC network. The number of such intact hepatocytes surrounded by complete BC filling by CF was counted in the area of interests. Approximately 0.05 mm² of the liver surface was analyzed in a single experiment for such evaluations.

Immunohistochemical Analyses of Subcellular Mrp2 Distribution

To evaluate Mrp2 in hepatocytes of the grafts, the liver samples were fixed, sliced, and stained with a monoclonal antibody M₂III-6, according to previous study (32). The antigen on the sections was visualized by phycoerythrin-conjugated anti-mouse IgG and observed through laser confocal microfluorography at 488 nm as described elsewhere (20, 30). In order to examine BC localization and hepatocellular internalization of Mrp2, the sections were double immunostained with a monoclonal antibody against ZO-1, another marker expressed in hepatocellular junction (33). To determine changes in the protein distribution in a semi-quantitative manner, single-stained microfluorographs of Mrp2 were converted as monochrome 8-bit images (14). The gray levels (1-256) were measured at both cytoplasmic and canalicular domains in a single hepatocytes. At least 5 different sites for each domain were chosen in a single cell to calculate the relative values of cytoplasmic intensities versus the corresponding canalicular intensities. Such a measurement was carried out in 40-60 hepatocytes in 4 different grafts to construct histograms of the percentage cytoplasmic intensities of Mrp2-associated immunoreactivities, being defined as % I-Mrp2(cyt/bc). The elevation of this index represented an increase in the Mtp2 internalization. The histograms were compared among the control grafts and those exposed to cold ischemia with and without the KC-depleting procedure in the presence or absence of the TXA₂ synthase inhibitor.

In order to examine differences in Mrp2 expression in the whole liver grafts among the groups, Western blot analyses were carried out using the same monoclonal antibody. We also investigated alterations in oxidative modification of Mrp2 by immunoprecipitating the protein by the antibody M₂III-6 to follow Western analyses by an anti-acrolein monoclonal antibody (5F6) (34, 35).

Statistical Analyses

The statistical significance of data among different experimental groups was determined by one-way ANOVA and Fischer's multiple comparison test. P<0.05 was considered significant.

Results

Storage Time-Dependent Reduction of Bile Output in Liver Grafts.

To test the viability of liver grafts, the release of lactate dehydrogenase in the venous perfusate was measured as an index of cell lysis. As seen in Table 1, the grafts exposed to cold ischemia for less than 24 hrs did not display any notable elevation of lactate dehydrogenase.

TABLE 1
Effect of the duration of cold preservation on the release
of LDH in the venous perfusate of the grafts.

| Length of cold storage (hrs) | 5 min-R | 60 min-R |
|---|---|---|
| Control | 20 ± 6 | 35 ± 11 |
| 8 | 31 ± 7 | 25 ± 9 |
| 16 | 21 ± 8 | 34 ± 6 |
| 24 | 45 ± 8 | 43 ± 22 |
| 48 | 878 ± 377* | 1206 ± 719* |

Data represent means ± SE of measurements (mIU/min/g liver) from the grafts at the onset (0 min) and 60 min after the start of reperfusion (0-24 hrs; n = 5, 48 hrs; n = 3).
*P < 0.05 as compared with the value in other groups.

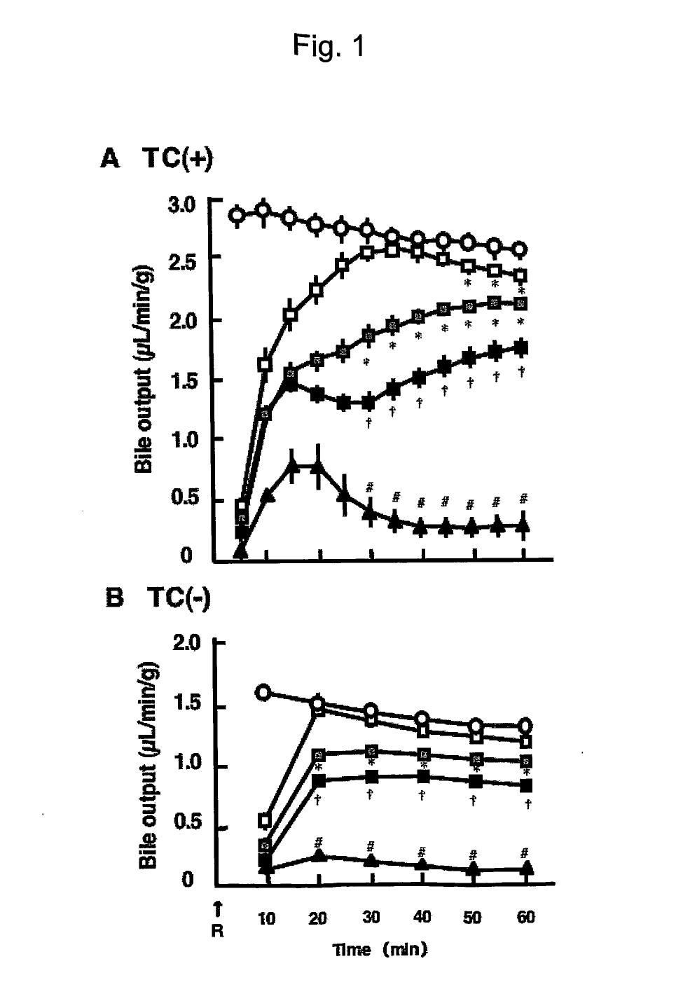

At 48 hrs, the lactate dehydrogenase release became evident at being and end of the 60 min reperfusion, showing that necrotic cell death was undetectable in cold ischemic grafts exposed to less than 24 hrs under the current experimental conditions. FIG. 1 illustrates time courses of bile output as a function of reperfusion time in grafts undergoing varied lengths of cold ischemia. As seen in Panel A where sodium taurocholate was added, the grafts exposed to 8-hr ischemia increased their output to the level comparable to that in the controls at 30 min but decreased it at 50-60 min after the initial reperfusion. In the grafts exposed to prolonged cold ischemia for 16-48 hrs, such a reduction of the output became further evident. Panel B of FIG. 1 showed the time course of bile recovery monitored in the absence of sodium taurocholate in the perfusate. As seen, the groups treated with cold ischemia for longer than 16 hrs displayed significant decreases in the output.

Alterations in Biliary Excretion of Glutathione and Bilirubin in Post-Cold Ischemic Livers

Observation of a significant decrease in bile flow in the grafts undergoing 16- and 24-hr cold ischemia led us to determine bile constituents responsible for cholestatic changes. FIG. 2 illustrated data of bile constituents measured at 20 min after the onset of reperfusion that were plotted as a function of storage time for cold ischemia. As seen, concentrations of bile salts did not exhibit any significant reduction in any length of storage time, while those of phospholipids displayed notable reduction in both concentrations and fluxes in the group exposed to 8- to 24-hr cold ischemia (FIGS. 2A and 2B). Considering that phospholipids are primarily excreted from hepatocytes into biliary compartment, these data suggest the presence of hepatocellular dysfunction in the grafts stored for longer than 8 hrs.

We next examined biliary excretion of GSH under monitoring its tissue content (FIG. 2C). Biliary concentrations in GSH were significantly reduced at 8 hrs and declined as a function of the cold storage time. We then examined hepatic content of GSH: as seen in FIG. 2D, hepatic GSH content did not change before and after 20-min reperfusion. In the grafts stored for 16 hrs, the content apparently increased as a result of the use of University of Wisconsin solution containing GSH. Upon 20-min reperfusion, however, the content was rapidly repressed to the control level as GSH was removed from circulation, showing that the 20-min reperfusion following 16-hr cold ischemia does not change the basal GSH content in the grafts. These results suggest that the decrease in biliary GSH excretion in the post-cold ischemic livers results from impairment of its transport to bile rather than from its reduction in the grafts, so far as the storage time was shorter than 16 hrs. Since GSH is excreted through Mrp2, we next examined alterations in biliary concentrations of BR-IXα, a bile pigment excreted through the same transporter. As seen in FIG. 2E, the biliary concentration of BR-IXα in the initial 20-min reperfusion was significantly elevated in the 8-hr ischemic group and became maximum in the 16-hr storage group. Finally, in the grafts undergoing 24-hr cold ischemia, initial concentrations of BR-IXα were abruptly decreased. FIG. 2F illustrates the ability of the grafts to eliminate endogenous BR-IXα into bile. As seen in the control grafts, hepatic content of this bile pigment significantly decreased within the initial 20-min perfusion. On the other hand, the same duration of reperfusion did not cause such a decrease in the 16-hr treated grafts. These results suggest that the ability of the 16-hr grafts to generate BR-IXα de novo surpasses their capacity to excrete the pigment into bile.

Global and Local Assessment of Mrp2 Function by Carboxyfluorescein Exclusion.

Alteration in biliary excretion of GSH and BR-IXα raised a possibility that the ability of Mrp2 to eliminate these organic anions from hepatocytes could be impaired in grafts exposed to prolonged cold ischemia. However, since initial amounts of glutathione and BR-IXα were different among groups, measuring biliary excretion of these endogenous anions did not allow us to make a fair comparison of the organic anion-excreting ability of the grafts. To overcome this difficulty, the grafts were loaded with CF, an exogenous organic anion, and its elimination from hepatocytes into bile was examined. As seen in the left panels of FIG. 3A, hepatocellular CF loading appeared comparable among the grafts exposed to different lengths (0-24 hrs) of cold ischemia. This was confirmed by the fluorescence intensitometry in FIG. 3C, indicating that the hepatocytes were viable. This was also consistent with results showing no notable release of LDH (Table 1). Immediately after the removal of probenecid, an inhibitor of Mrp2, CF loaded in hepatocytes was rapidly excreted into bile canaliculi, forming honeycomb networks over the lobule within 10 min (the middle column in FIG. 3A). At 25 min (the right column), little fluorescence inside the cytoplasm became detectable, if any. In grafts that have undergone cold-ischemia reperfusion, two major changes in biliary CF excretion have occurred: retardation of hepatocellular dye exclusion as judged by an elevation of the basal fluorescence at 25 min, and disappearance and deformation of bile canalicular networks as indicated in micrographs collected at 10 min. These changes became evident in grafts exposed to extended cold ischemia for 24 hrs (the bottom row in FIG. 3A).

Careful scanning at the site of bile canaliculi in these microfluorographs captured at 10 min showed minimal but notable changes in structure of the canalicular networks. As seen in FIG. 3B, the number of hepatocytes that were completely surrounded by CF-filled BC decreased as duration of cold ischemia increased. Such a reduction of polygons became readily apparent in the grafts stored for 16 hrs. Since the initial CF loading in hepatocytes was comparable in a range between 0 and 24 hrs of the ischemic duration, morphological changes in BC appeared to occur initially at 16-hr cold ischemia. In other words, grafts exposed to 8-hr cold ischemia did not exhibit any significant changes in morphology of BC networks.

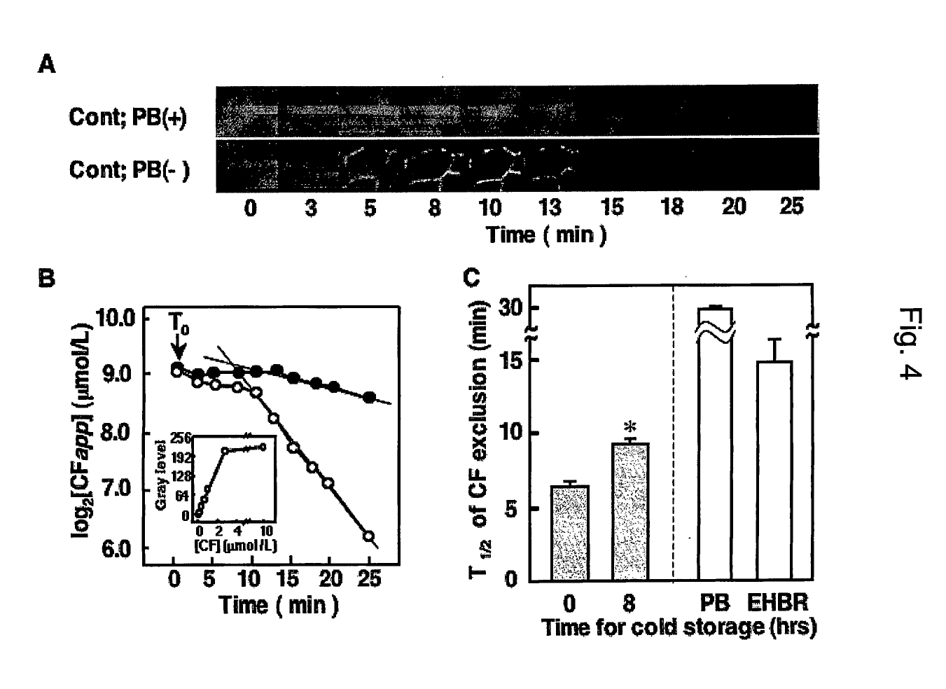

The retardation of the CF exclusion was further examined in a quantitative manner by monitoring temporal alterations in the fluorescence in hepatocytes of the grafts (FIG. 4A). As seen, when probenecid was perfused continuously, the dye stayed in the cells, exhibiting a slight decline without showing canalicular excretion. As plotted in FIG. 4B, gray levels measured at hepatocytes allowed us to determine the T_{1/2} of the CF exclusion from hepatocytes. FIG. 4C illustrates T_{1/2} values among the groups. In the absence of probenecid, T_{1/2} was approximately 6 min, while in the presence of probenecid the decay was substantially slowed suggesting near complete inhibition of Mrp2 transport. As seen, T_{1/2} of the 8-hr cold ischemic graft to excrete CF was significantly greater, ranging at the midway between the control value and that measured in livers of EHBR. It could be speculated that the smaller T_{1/2} values in this mutant species as compared with the probenecid-treated group is due to compensatory excretion of the dye through Mrp3 into the sinusoidal space (36).

Effects of KC Depletion on Biliary CF Excretion in 8-hr Cold Ischemic Grafts.

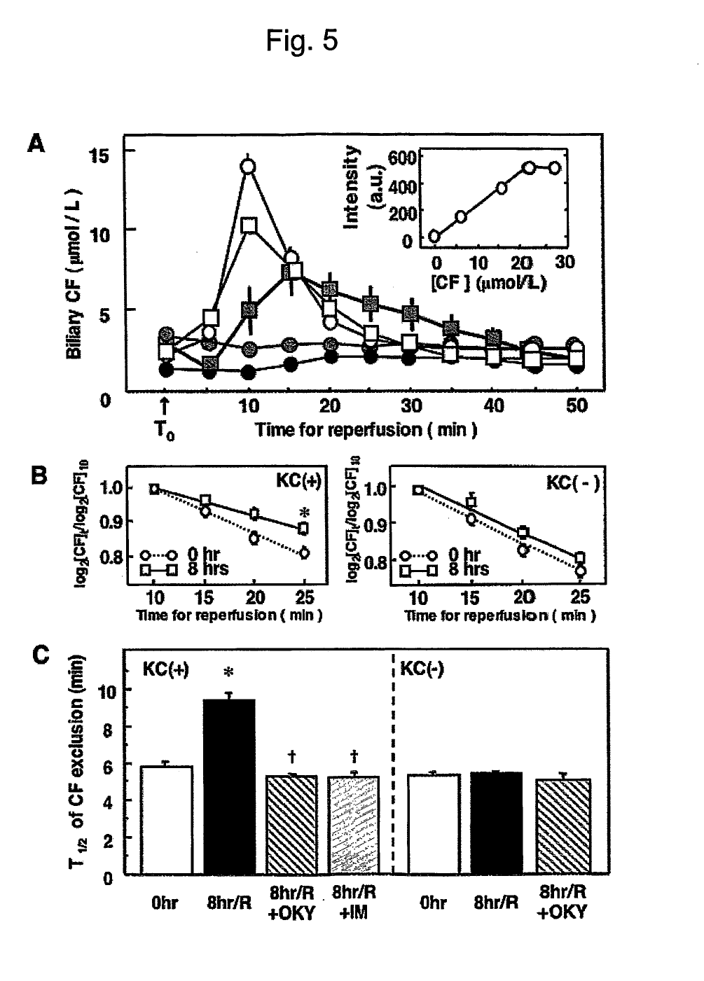

We then attempted to evaluate the ability of the 8-hr cold ischemic grafts as a whole to excrete CF. To this end, the T_{1/2} values for the dye exclusion were determined (FIG. 5). After removal of probenecid (T₀ in FIG. 5A), the CF concentrations in bile transiently increased and gradually returned to the basal level. Such a transient increase was not observed either in the presence of probenecid (shaded circles), or in the grafts isolated from EHBR (closed circles), suggesting that Mrp2 is responsible for biliary CF excretion. When the CF exclusion was analyzed in the whole liver grafts stored for 8 and 16 hrs, the decay appeared to be slower than that in the controls. Using the data collected from the 8-hr ischemic grafts, logarythmic values of the CF concentrations versus those at the peak (10 min) in bile samples were re-plotted as a function of reperfusion time (FIG. 5B). The T_{1/2} values of the dye exclusion were then compared between the grafts treated with and without the KC depletion. As seen, in the livers untreated with LDD [KC(+)], the 8-hr cold ischemia exhibited prolonged T_{1/2} values than the control. Such a difference in T_{1/2} between the two groups completely disappeared in the KC-depleting grafts. To be noted was that in the non-cold ischemic control livers, the KC-depleting procedure by itself did not alter the T_{1/2} values indicating that the ameliorating effect of the KC depletion became evident only when the grafts experienced cold ischemia-reperfusion.

To reveal mechanisms by which KC in the 8-hr cold ischemic grafts caused prolonged CF excretion through mrp2, we examined involvement of thromboxanes, a major prostanoid released from activated KCs under post-cold-ischemic conditions (37, 38). To this end, effects of OKY-046, an inhibitor of TXA₂ synthase, were examined. As seen in FIG. 5C, application of this reagent at 240 μmol/L to the storage and rinse solutions abolished an increase in T_{1/2} values of the CF exclusion almost completely. Since the TXA₂ synthase inhibitor might increase availability of arachidonic acid to synthesize other prostanoids (e.g. PGE₂ and PGF_{2α}, we examined if indomethacin (IM), an inhibitor that suppresses all the prostanoids produced via the cyclooxygenase pathway including TXs, could attenuate or aggravate the T_{1/2} values. As shown, this inhibitor also attenuated the prolonged T_{1/2} values almost completely. Furthermore, effects of OKY-046 on the 8-hr cold ischemic grafts became disappeared when KC was depleted, suggesting that the involvement of TXs is KC-dependent.

Intracellular Re-Localization of Mrp2 by 8-hr Cold Ischemia and its Attenuation by KC Depletion.

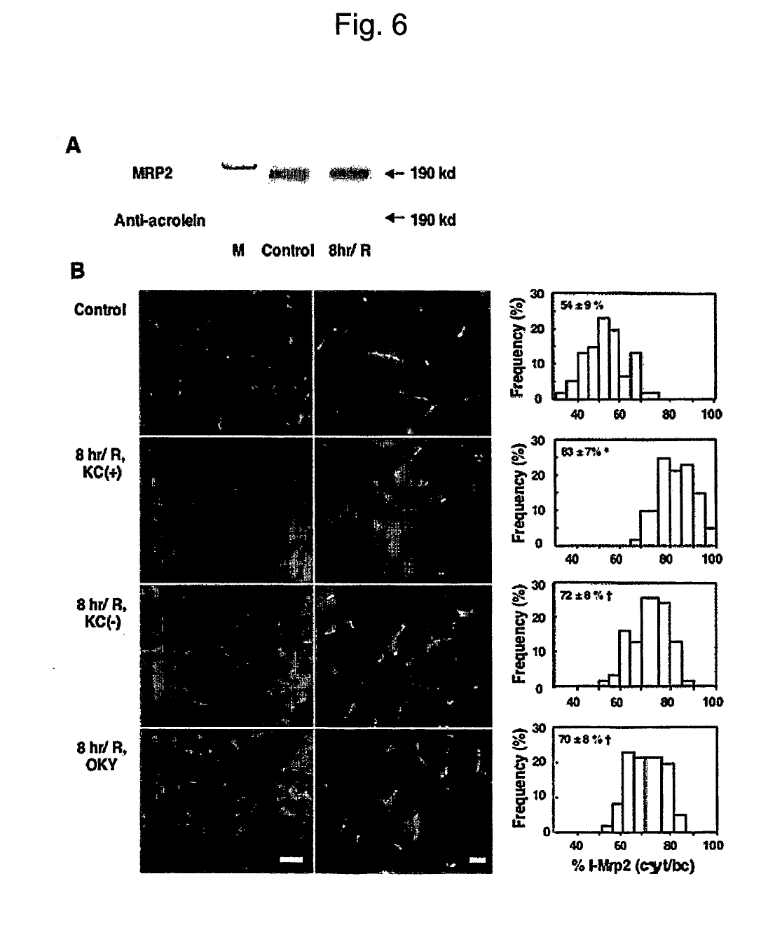

Since KC is known to down-regulate Mrp2 in endotoxin-treated livers (39), we examined if such a change could be involved in the mechanisms for the dysfunction of the transporter. As shown by Western blot analyses, amounts of Mrp2 protein was unchanged in the 8-hr cold ischemic grafts (FIG. 6A) as well as in the 24-hr ischemic grafts (data not shown). We also examined if the protein by itself was oxidatively modified as a consequence of post-ischemic oxidative insults. However, no apparent changes were found as judged by immunoprecipitation using the anti-acrolein antibody. It was then examined if hepatocellular localization of the protein is modified in the 8-hr cold ischemic grafts. As seen in FIG. 6B, its localization in BC was markedly reduced, while the background fluorescence in cytoplasm of hepatocytes was elevated in the 8-hr ischemia-reperfused grafts. As seen in the lower panels, double immunostaining with ZO-1 (green) revealed that co-localization of Mrp2 (red) in BC was markedly disrupted, while its intensities in the cytoplasm were elevated, suggesting the internalization of the protein. Such changes were attenuated in the grafts treated with the KC depletion or with OXY-046. As the % I-Mrp2 (cyt/bc) values, an index for internalization, indicated that the 8-hr cold ischemia-reperfusion significantly enhanced the Mrp2 internalization and that treatment with KC depletion or with blockade of TXA₂ synthase improved bile canalicular re-localization of the transporter.

We determined differences in hepatic contents of ATP and cAMP in the KC(+) and KC(−) grafts after the 8-hr cold ischemia-reperfusion, but without any notable significance between the two groups (3.4±0.9 vs 2.9±1.1 μmol/g liver in ATP content, and 8.9±1.0 vs 9.2±0.5 pmol/g liver in cAMP content, respectively). These results suggest that amelioration of intracellular retrieval of this ATP-binding protein by the KC depletion did not result from alterations in tissue contents of ATP and cAMP.

### INDUSTRIAL APPLICABILITY

The present invention is useful for a revival of preclinical reagents that were previously dropped off for its organ toxicity. Overall, detailed mechanisms for the toxicity of compounds indicating hepatotoxicity upon routine clinical chemistry remain largely unknown. If such compounds are examined in detail through the present intervension and turn out to block only transporter function without affecting cell viability, minor modification of the chemical structure would be effective to be applied for clinical use. (2) Application of the present method to immunodeficient mice implanted with human cancer cells allows us to examine specificity of any types of drug delivery and accumulation to tumor cell aggregates in vivo or to intact tissues. Such an application is extremely useful to examine the delivery and elimination of anti-cancer reagents in in-vivo implanted cancers, since function of transporters such as mrp2 is an important determinant of the drug resistance of tumors. Application of preclinical experiments for screening potentials of anti-cancer and anti-thrombotic reagents.

### REFERENCES

1. Ploeg R J, D'Alessandro A M, Knechtle S J, Stegall M D, Pirsch J D, Hoffmann R M, Sasaki T, et al. Risk factors for primary dysfunction after liver transplantation—a multivariate analysis. Transplantation 1993;55:807-813.

2. Deschenes M, Belle S H, Krom R A, Zetterman R K, Lake J R. Early allograft dysfunction after liver transplantation: a definition and predictors of outcome. National Institute of Diabetes and Digestive and Kidney Diseases Liver Transplantation Database. Transplantation 1998;66:302-310.

3. Porte R J, Ploeg R J, Hansen B, van Bockel J H, Thorogood J, Persijn G G. Hermans J, et al. Long-term graft survival after liver transplantation in the UW era: late effects of cold ischemia and primary dysfunction. European Multicentre Study Group. Transpl Int 1998;11 Suppl 1:S164-167.

4. Adam R, Bismuth H, Diamond T, Ducot B, Morino M, Astarcioglu I, Johann M, et al. Effect of extended cold ischemia with UW solution on graft function after liver transplantation. Lancet 1992;340: 1373-1376.

5. Jaeschke H. Preservation injury: mechanisms, prevention and consequences. J Hepatol 1996;25:774-780.

6. Caldwell-Kenkel J C, Currin R T, Tanaka Y, Thurman R G, Lemasters J J. Reperfusion injury to endothelial cells following cold ischemic storage of rat livers. Hepatology 1989; 10:292-299.

7. Caldwell-Kenkel J C, Currin R T, Tanaka Y, Thurman R G, Lemasters J J. Kupffer cell activation and endothelial cell damage after storage of rat livers: effects of reperfusion. Hepatology 1991;13:83-95.

8. Lemasters J J, Thurman R G. Reperfusion injury after liver preservation for transplantation. Annu Rev Pharmacol Toxicol 1997;37:327-338.

9. Clavien P A. Sinusoidal endothelial cell injury during hepatic preservation and reperfusion. Hepatology 1998;28:281-285.

10. Sindram D, Porte R J, Hoffman M R, Bentley R C, Clavien P A. Platelets induce sinusoidal endothelial cell apoptosis upon reperfusion of the cold ischemic rat liver. Gastroenterology 2000;1 18:183-191.

11. Arai M, Mochida S, Ohno A, Fujiwara K. Blood coagulation in the hepatic sinusoids as a contributing factor in liver injury following orthotopic liver transplantation in the rat. Transplantation 1996;62:1398-1401.

12. Clavien P A, Harvey P R, Sanabria J R, Cywes R, Levy G A, Strasberg S M. Lymphocyte adherence in the reperfused rat liver: mechanisms and effects. Hepatology 1993;17:131-142.

13. Vajdova K, Smrekova R, Mislanova C, Kukan M, Lutterova M. Cold-preservation-induced sensitivity of rat hepatocyte function to rewarming injury and its prevention by short-term reperfusion. Hepatology 2000;32:289-296.

14. Kumamoto Y, Suematsu M, Shimazu M, Kato Y, Sano T, Makino N, Hirano K I, et al. Kupffer cell-independent acute hepatocellular oxidative stress and decreased bile formation in post-cold-ischemic rat liver. Hepatology 1999;30:1454-1463.

15. Imamura H, Brault A, Huet P M. Effects of extended cold preservation and transplantation on the rat liver microcirculation. Hepatology 1997;25:664-671.

16. Williams J W, Vera S, Peters T G, Van Voorst S, Britt L G, Dean P J, Haggitt R, et al. Cholestatic jaundice after hepatic transplantation. A nonimmunologically mediated event. Am J Surg 1986;151:65-70.

17. Suzuki H, Suematsu M, Ishii H, Kato S, Miki H, Mori M, Ishimura Y, et al. Prostaglandin E1 abrogates early reductive stress and zone-specific paradoxical oxidative injury in hypoperfused rat liver. J Clin Invest 1994;93:155-164.

18. Suematsu M, Goda N, Sano T, Kashiwagi S, Egawa T, Shinoda Y, Ishimura Y. Carbon monoxide: an endogenous modulator of sinusoidal tone in the perfused rat liver. J Clin Invest 1995;96:2431-2437.

19. Kyokane T, Norimizu S, Taniai H, Yamaguchi T, Takeoka S, Tsuchida E, Naito M, et al. Carbon monoxide from heme catabolism protects against hepatobiliary dysfunction in endotoxin-treated rat liver. Gastroenterology 2001;120:1227-1240.

20. Goda N, Suzuki K, Naito M, Takeoka S, Tsuchida E, Ishimura Y, Tamatani T, et al. Distribution of heme oxygenase isoforms in rat liver. Topographic basis for carbon monoxide-mediated microvascular relaxation. J Clin Invest 1998; 101:604-612.

21. Shiomi M, Wakabayashi Y, Sano T, Shinoda Y, Nimura Y, Ishimura Y, Suematsu M. Nitric oxide suppression reversibly attenuates mitochondrial dysfunction and cholestasis in endotoxemic rat liver. Hepatology 1998;27:108-115.

22. Ishiguro S, Arii S, Monden K, Fujita S, Nakamura T, Niwano M, Harada T, et al. Involvement of thromboxane A₂-thromboxane A₂ receptor system of the hepatic sinusoid in pathogenesis of cold preservation/reperfusion injury in the rat liver graft. Transplantation 1995; 59: 957-961.

23. Jaeschke H, Smith C V, Mitchell J R. Reactive oxygen species during ischemia-reflow injury in isolated perfused rat liver. J Clin Invest 1988;81:1240-1246.

24. Yamaguchi T, Wakabayashi Y, Tanaka M, Sano T, Ishikawa H, Nakajima H, Suematsu M, et al. Taurocholate induces directional excretion of bilirubin into bile in perfused rat liver. Am J Physiol 1996;270:G1028-1032.

25. Izumi Y, Yamazaki M, Shimizu S, Shimizu K, Yamaguchi T, Nakajima H. Anti-bilirubin monoclonal antibody. II. Enzyme-linked immunosorbent assay for bilirubin fractions by combination of two monoclonal antibodies. Biochim Biophys Acta 1988;967:261-266.

26. Mor-Cohen R, Zivelin A, Rosenberg N, Shani M, Muallem S, Seligsohn U. Identification and functional analysis of two novel mutations in the multidrug resistance protein 2 gene in Israeli patients with Dubin-Johnson syndrome. J Biol Chem 2001 ;276:36923-36930.

27. van der Kolk D M, de Vries E Q Noordhoek L, van den Berg E, van der Pol M A, Muller M, Vellenga E. Activity and expression of the multidrug resistance proteins P-glycoprotein, MRP1, MRP2, MRP3 and MRP5 in de novo and relapsed acute myeloid leukemia. Leukemia 2001;15:1544-1553.

28. Courtois A, Payen L, Lagadic D, Guillouzo A, Fardel O. Evidence for a multidrug resistance-associated protein 1 (MRP1)-related transport system in cultured rat liver biliary epithelial cells. Life Sci 1999;64:763-774.

29. Suematsu M, Suzuki H, Ishii H, Kato S, Yanagisawa T, Asako H, Suzuki M, et al. Early midzonal oxidative stress preceding cell death in hypoperfused rat liver. Gastroenterology 1992;103:994-1001.

30. Kajimura M, Shimoyarna M, Tsuyama S, Suzuki T, Kozaki S, Takenaka S, Tsubota K, et al. Visualization of gaseous monoxide reception by soluble guanylate cyclase in the rat retina. Faseb J 2003;17:506-508.

31. Hayashi S, Takamiya R, Yamaguchi T, Matsumoto K, Tojo S J, Tamatani T, Kitajima M, et al. Induction of heme oxygenase-1 suppresses venular leukocyte adhesion elicited by oxidative stress: role of bilirubin generated by the enzyme. Circ Res 1999;85:663-671.

32. Mottino A D, Cao J, Veggi L M, Crocenzi F, Roma M G, Vore M. Altered localization and activity of canalicular Mrp2 in estradiol-17beta-D-glucuronide-induced cholestasis. Hepatology 2002;35:1409-1419.

33. Kubitz R, D'Urso D, Keppler D, Haussinger D. Osmodependent dynamic localization of the multidrug resistance protein 2 in the rat hepatocyte canalicular membrane. Gastroenterology 1997;113:1438-1442.

34. Uchida K, Kanematsu M, Sakai K, Matsuda T, Hattori N, Mizuno Y, Suzuki D, et al. Protein-bound acrolein: potential markers for oxidative stress. Proc Natl Acad Sci USA 1998;95:4882-4887.

35. Tanaka N, Tajima S, Ishibashi A, Uchida K, Shigematsu T. Immunohistochemical detection of lipid peroxidation products, protein-bound acrolein and 4-hydroxynonenal protein adducts, in actinic elastosis of photodamaged skin. Arch Dermatol Res 2001;293:363-367.

36. Ogawa K, Suzuki H, Hirohashi T, Ishikawa T, Meier P J, Hirose K, Akizawa T, et al. Characterization of inducible nature of MRP3 in rat liver. Am J Physiol Gastrointest Liver Physiol 2000;278:G438-446.

37. Gyenes M, and De Groot H. Prostanoid release by Kupffer cells upon hypoxia-reoxygenation: role of pHi and Ca²⁺. Am J Physiol 1993; 264: G535-G540.

38. Johnston D E, Peterson M B, Mion F, Beminger R W, Jefferson D M. Synthesis and degradation of eicosanoids in primary rat hepatocyte cultures. Pro staglandins Leukot Essent Fatty Acids 1991; 43: 119-132.

39. Nakamura J, Nishida T, Hayashi K, Kawada N, Ueshima S, Sugiyama Y, Ito T, et al. Kupffer cell-mediated down regulation of rat hepatic CMOAT/MRP2 gene expression. Biochem Biophys Res Commun 1999;255:143-149.

40. Roelofsen H, Soroka C J, Keppler D, Boyer J L. Cyclic AMP stimulates sorting of the canalicular organic anion transporter (Mrp2/cMoat) to the apical domain in hepatocyte couplets. J Cell Sci 1998; 111 (Pt8):1137-1145.

41. Kipp H, Arias I M. Intracellular trafficking and regulation of canalicular ATP-binding cassette transporters. Semin Liver Dis 2000;20:339-351.

42. Horton A A, Wood J M. Prevention of thromboxane B2-induced hepatocyte plasma membrane bleb formation by certain prostaglandins and a proteinase inhibitor. Biochim Biophys Acta 1990;1022:319-324

43. Kato Y, Shimazu M, Kondo M, Uchida K, Kumamoto Y, Wakabayashi G, Kitajima M, et al. Bilirubin rinse: A simple protectant against the rat liver graft injury mimicking heme oxygenase-1 preconditioning. Hepatology 2003;3 8:364-373.

All documents are hereby incorporated by reference for all purposes.

1. A method of analyzing organ or tissue injury, comprising the following steps of:
(a) labeling an organ or a tissue with dye;
(b) obtaining multiple indices involving xenobiotic metabolism and/or cell condition of said organ or tissue; and
(c) analyzing the organ or tissue injury from said indices.

2. The method of claim 1, wherein the organ or tissue is at least one selected from the group consisting of liver, kidney, lung, pancreas and gastrointestinal tracts.

3. The method of claim 1, where the step (b) further comprises a step of obtaining microanatomical orientation of vascular system and/or excretion pathways.

4. The method of claim 1, wherein the analysis is carried out visually and/or quantitatively.

5. The method of claim 1, wherein the cell condition is at least one selected from the group consisting of cell viability, cell injury, molecular transport, and mitochondrial function.

6. A method of evaluating drug toxicity, comprising the following steps of:
(a) labeling an organ or a tissue with dye;
(b) applying a test drug to said organ or tissue;
(c) obtaining multiple indices involving xenobiotic metabolism and/or cell condition of said organ or tissue;
(d) analyzing the organ or tissue injury from said indices; and
(e) evaluating whether or not the drug have a toxicity to said organ or tissue.

7. The method of claim 6, wherein the organ or tissue is at least one selected from the group consisting of liver, kidney, lung, pancreas and gastrointestinal tracts.

8. The method of claim 6, where the step (b) further comprises a step of obtaining microanatomical orientation of vascular system and/or excretion pathways.

9. The method of claim 6, wherein the analysis is carried out visually and/or quantitatively.

10. The method of claim 6, wherein the cell condition is at least one selected from the group consisting of cell viability, cell injury, transport of molecules in and around cells, and generation of biologically active compounds, blood flow, and tissue oxygenation.

11. The method of claim 2, where step (b) further comprises a step of obtaining microanatomical orientation of vascular system and/or excretion pathways.

12. The method of claim 2, wherein the analysis is carried out visually and/or quantitatively.

13. The method of claim 2, wherein the cell condition is at least one of selected from the group consisting of cell viability, call injury, molecular transport, and mitochondrial function.

14. The method of claim 7, where step (b) further comprises a step of obtaining microanatomical orientation of vascular system and/or execrteion pathways.

15. The method of claim 7, wherein the analysis is carried out visually and/or quantitatively.

16. The method of claim 7, wherein the cell condition is at least one of selected from the group consisting of cell viability, call injury, molecular transport, and mitochondrial function.
